(19) United States
(12) Patent Application Publication
(10) Pub. No.: US 2024/0252023 A1
(43) Pub. Date: Aug. 1, 2024

(54) MEDICAL DEVICE PORT CONNECTORS

(71) Applicant: Saban Ventures Pty Limited, Macquarie Park, NSW (AU)

(72) Inventors: Nishant RANDHAWA, Grantham Farm, NSW (AU); Gavin SPARGO, Box Hill, NSW (AU); Panagiotis THRASOU, St Marys, NSW (AU); David Anthony PIDCOCK, Alexandria, NSW (AU)

(21) Appl. No.: 18/566,304

(22) PCT Filed: Jun. 3, 2022
(86) PCT No.: PCT/AU2022/050547

§ 371 Date: Dec. 1, 2023

(30) Foreign Application Priority Data
Jun. 3, 2021 (AU) 2021901655

(51) Int. Cl. A61B 1/00; A61B 1/12

(52) U.S. Cl. CPC A61B 1/00128; A61B 1/125

(57) ABSTRACT

A suction and air/water port connector for a medical device includes a body, a gripping portion coupled to the body and a force generator configured to be at least partially seated within the body. The locking member is configured to removably couple to an exterior surface of a suction port and an air/water port of the medical device to define a locked position and an unlocked position. When in the locked position, movement of the body relative to the suction port and the air/water port is inhibited and a locking force is exerted by the force generator in an axial direction against the suction port and the air/water port to create a liquid-tight seal against the suction port and the air/water port. When in the unlocked position, the locking member is configured to be removed from the suction port and the air/water port.

### CROSS REFERENCE TO RELATED APPLICATIONS

This application claims the benefit of and priority to Australian Patent Application No. 2021901655, filed Jun. 3, 2021. The entire contents of said application is hereby incorporated by reference.

### BACKGROUND

Any discussion of the prior art throughout the specification should in no way be considered as an admission that such prior art is widely known or forms part of common general knowledge in the field.

Often times, reusable medical devices need to be cleaned and/or sterilized and/or disinfected prior to use. Many medical devices—such as endoscopes—have one or more lumens, and cleaning of such devices with lumens can be accomplished via flushing the respective lumen(s). Current methods and systems used to clean such medical devices involve several manual cleaning steps, are very time consuming, and yield varying results. Accordingly, there remains numerous challenges in the field of medical device cleaning systems and methods.

### BRIEF SUMMARY

According to a first aspect of the present invention, there is provided a suction and air/water port connector for a medical device, comprising:
a body assembly;
a gripping portion coupled to the body assembly;
a force generator configured to be at least partially seated within the body assembly;
one or more sealing cups at least partially housed within the body assembly; and
a locking member operatively coupled to the body assembly and configured to removably couple to an exterior surface of a suction port and an air/water port of the medical device to define a locked position and an unlocked position,
wherein in the locked position, movement of the body assembly relative to the suction port and the air/water port is inhibited and a locking force is exerted by the force generator in an axial direction against the suction port and the air/water port to create a liquid-tight seal against the suction port and the air/water port, and
wherein in the unlocked position, the locking member is configured to be removed from the suction port and the air/water port.

According to a second aspect of the present invention, there is provided a method of manufacturing a suction and air/water port connector for a medical device, comprising:
structuring a body assembly to,
   couple to a gripping portion,
   at least partially surround a force generator configured to generate a locking force, and
   at least partially house one or more sealing cups;
structuring a locking member to operatively couple to the body assembly and to removably couple to an exterior surface of a suction port and an air/water port of the medical device to define a locked position and an unlocked position; and
exerting the locking force in an axial direction against the suction port and the air/water port to create a liquid-tight seal against the suction port and the air/water port when in the locked position.

According to a third aspect of the present invention, there is provided a port connector for a medical device, comprising:
a connector body including one or more gripping elements;
a receptacle configured to be at least partially housed within the connector body and define a fluid path at least partially through the connector body;
a first shuttle plate operatively coupled to the connector body and configured to move relative to the connector body; and
a second shuttle plate operatively coupled to the connector body and configured to move relative to the connector body,
wherein the first shuttle plate and the second shuttle plate are configured to be biased against and surround an exterior surface of the port,

wherein a biasing force is exerted against the receptacle in a direction towards the biopsy port to establish a liquid-tight seal against an exterior surface of the biopsy port.

According to a fourth aspect of the present invention, there is provided a port connector for a medical device, comprising:
a housing configured to rotatably couple to an outer surface of an auxiliary port; and
a barb supported by and sealed against the housing, wherein the barb defines a fluid path at least partially through the housing and is configured to rotate relative to the housing,
wherein the housing is configured to rotate independent of the barb to engage the auxiliary port and create a liquid-tight seal against the auxiliary port, and wherein the barb is enabled to swivel freely during engagement and disengagement with the auxiliary port.

Endoscope connectors are high end use products with lot of human interactions, and one challenge has been the development of connectors that are both user friendly and form the compliant seals required to quickly and efficiently clean endoscopes and other medical devices at pressures including and in excess of 24 psi. Hence, the disclosed connectors are designed for ease of use while achieving best functional outcomes during a cleaning cycle. As used herein, a “cleaning cycle” is meant to include a cleaning and/or disinfection and/or sterilization process. In order to address the challenge of cleaning and/or disinfection and/or sterilizing reusable medical devices such as endoscopes between patients, the inventors have developed connectors for connecting the ports of a medical device containing lumen(s) to a cleaning and/or disinfection and/or sterilization source or device. For example, embodiments of the disclosure can operate as connectors configured to engage with the suction and air/water ports, the biopsy port, and/or the auxiliary port of an endoscope.

Optionally, one or more of these connectors may be provided in a kit and/or may be provided individually. The connectors comprise, for example, a suction and air/water port connector, a biopsy port connector and an auxiliary port connector.

In one embodiment of a suction and air/water port connector, the suction and air/water port connector is structured to interface with an endoscope channel cleaner device and an endoscope In one embodiment, the suction and air/water port connector's small size facilitates fitting and sealing it onto the suction and air/water cylinders. In one embodiment, the suction and air/water port connector is configured for simple insertion and removal actions, e.g., a push and twist mechanism that engages under the cylinder lips to securely attach the connector to the ports. One embodiment of the suction and air/water port connector comprises a locking spring or force generator that provides sealing force between multiple components of the connector, so fluid may flow through each cylinder. One of the major advantages of this connector is ease of use, where locking spring has been custom designed and tuned to provide sealing force while keeping user acceptable forces for insertion and removal actions. Other embodiments of the suction and air/water port connector are structured to interface with different medical devices while still providing some, if not all of the benefits and advantages described, including provide robust attachment and sealing to the medical device and the corresponding medical device cleaning device.

One embodiment of a biopsy connector or biopsy port connector is structured to interface with an endoscope channel cleaner and an endoscope. In an embodiment, the biopsy connector comprises dual shuttles, and is configured for simple insertion and removal actions, e.g., a push and latch mechanism that engages under the biopsy port lip to for secure attachment. One embodiment of the biopsy port connector comprises a locking spring or force generator that provides sealing force between multiple components of the connector, so fluid may flow through biopsy cylinder. Other embodiments of the biopsy port connector are structured to interface with different medical devices while still providing some, if not all of the benefits and advantages described, including provide robust attachment and sealing to the medical device and the corresponding medical device cleaning device. As used herein, the terms “cleaning device” or “medical device cleaning device” are meant to include devices capable of cleaning and/or disinfection and/or sterilization.

One embodiment of an auxiliary connector or auxiliary port connector is structured to interface with an endoscope channel cleaner device and an endoscope. One embodiment of the auxiliary connector provides a means of screwing and unscrewing on the port while enabling swiveling of a hose barb and any externally connected hoses. In one embodiment, sealing is achieved via the radial lip seal inside the housing and the bottom seal which engages with the port directly. Other embodiments of the auxiliary port connector are structured to interface with different medical devices while still providing some, if not all of the benefits and advantages described, including provide robust attachment and sealing to the medical device and the corresponding medical device cleaning device.

In an embodiment, one or more port connectors are provided for connecting a reusable medical device to a medical device cleaning device. In the case where the reusable medical device is an endoscope, the one or more port connectors may include a suction air/water port connector configured to fluidly couple to an exterior surface of a suction port and an air/water port of the endoscope device. The one or more port connectors may further include a biopsy port connector configured to fluidly couple to an exterior surface of a biopsy port of the endoscope device. The one or more port connectors may further include an auxiliary port connector configured to fluidly couple to an exterior surface of an auxiliary port of the endoscope device. The suction air/water port connector, the biopsy port connector and the auxiliary connector are configured to enable cleaning and disinfection fluids to be automatically introduced into the suction port, the air/water port, the biopsy port and the auxiliary port to clean the endoscope device.

An embodiment of a suction and air/water port connector for a medical device includes a body, a gripping portion coupled to the body and a force generator configured to be at least partially seated within the body. One or more sealing cups are at least partially housed within the body and a locking member operatively coupled to the body. The locking member is configured to removably couple to an exterior surface of a suction port and an air/water port of the medical device to define a locked position and an unlocked position. When in the locked position, movement of the body relative to the suction port and the air/water port is inhibited and a locking force is exerted by the force generator in an axial direction against the suction port and the air/water port to create a liquid-tight seal against the suction port and the air/water port. When in the unlocked position, the locking member is configured to be removed from the suction port and the air/water port.

An embodiment of a method of manufacturing a suction and air/water port connector for a medical device includes structuring a body assembly to couple to a gripping portion, at least partially surround a force generator configured to generate a locking force, and at least partially house one or more sealing cups. A locking member is structured to operatively couple to the body assembly and to removably couple to an exterior surface of a suction port and an air/water port of the medical device to define a locked position and an unlocked position. The locking force is exerted in an axial direction against the suction port and the air/water port to create a liquid-tight seal against the suction port and the air/water port when in the locked position.

An embodiment of a port connector for a medical device includes a connector body including one or more gripping elements and a receptacle configured to be at least partially housed within the connector body to define a fluid path at least partially through the connector body. A first shuttle plate is operatively coupled to the body and configured to move relative to the body and a second shuttle plate is operatively coupled to the body and configured to move relative to the body. The first shuttle plate and the second shuttle plate are configured to be biased against and surround an exterior surface of the a biopsy port wherein a biasing force is exerted against the receptacle in a direction towards the port to establish a liquid-tight seal against an exterior surface of the port.

Another embodiment of a port connector for a medical device includes a housing configured to rotatably couple to an outer surface of a port and a barb supported by and sealed against the housing. The barb defines a fluid path at least partially through the housing and is configured to rotate relative to the housing. The housing is configured to rotate independent of the barb to engage the port and create a liquid-tight seal against the port. The barb is enabled to swivel freely during engagement and disengagement with the port.

The above embodiments are exemplary only.

Other embodiments as described herein are within the scope of the disclosed subject matter.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

### BRIEF DESCRIPTION OF THE SEVERAL VIEWS OF THE DRAWINGS

So that the manner in which the features of the disclosure can be understood, a detailed description may be had by reference to certain embodiments, some of which are illustrated in the accompanying drawings. It is to be noted, however, that the drawings illustrate only certain embodiments and are therefore not to be considered limiting of its scope, for the scope of the disclosed subject matter encompasses other embodiments as well. The drawings are not necessarily to scale, emphasis generally being placed upon illustrating the features of certain embodiments. In the drawings, like numerals are used to indicate like parts throughout the various views, in which:

FIG. 1A depicts one embodiment of an endoscope, in accordance with one or more aspects of the present disclosure;

FIG. 1B shows a close-up view of the suction and air/water ports of FIG. 1A, in accordance with one or more aspects of the present disclosure;

FIG. 1C shows a close-up view of the auxiliary and suction ports of FIG. 1A, in accordance with one or more aspects of the present disclosure;

FIG. 1D shows a close-up view of the air water bottle port of FIG. 1A, in accordance with one or more aspects of the present disclosure;

FIG. 1E shows a close-up view of the air-pipe port of FIG. 1A, in accordance with one or more aspects of the present disclosure;

FIG. 1F shows a close-up view of the biopsy port of FIG. 1A, in accordance with one or more aspects of the present disclosure;

FIG. 2A illustrates one embodiment of a suction and air/water port connector being lowered onto the suction port and the air/water port of an endoscope, in accordance with one or more aspects of the present disclosure;

FIG. 2B illustrates rotation the suction and air/water port connector knob shown in FIG. 2A to a locked position, in accordance with one or more aspects of the present disclosure;

FIG. 2C depicts the suction and air/water port connector of FIG. 2A locked in position onto the suction and air/water ports of an endoscope, in accordance with one or more aspects of the present disclosure;

FIG. 2D shows an exploded view of the suction and air/water port connector shown in FIG. 2A, in accordance with one or more aspects of the present disclosure;

FIG. 2E illustrates the knob assembly of the suction and air/water port connector shown in FIG. 2A, in accordance with one or more aspects of the present disclosure;

FIG. 2F illustrates a bottom view of the knob assembly of the suction and air/water port connector shown in FIG. 2A, in accordance with one or more aspects of the present disclosure.

FIG. 2G illustrates the main body of the suction and air/water port connector shown in FIG. 2A, in accordance with one or more aspects of the present disclosure;

FIG. 2H illustrates a cross-section of the main body of the suction and air/water port connector shown in FIG. 2A, in accordance with one or more aspects of the present disclosure;

FIG. 2I illustrates a sealing cup and channel separator pin of the suction and air/water port connector shown in FIG. 2A, in accordance with one or more aspects of the present disclosure;

FIG. 2J illustrates the sealing cup of the suction and air/water port connector shown in FIG. 2A, in accordance with one or more aspects of the present disclosure;

FIG. 2K illustrates a cross section of the sealing cup of the suction and air/water port connector shown in FIG. 2J, in accordance with one or more aspects of the present disclosure;

FIG. 2L illustrates positioning of one embodiment of a channel separator pin of the suction and air/water port connector shown in FIG. 2A, in accordance with one or more aspects of the present disclosure;

FIG. 2M illustrates positioning of another embodiment of a channel separator pin of the suction and air/water port connector shown in FIG. 2A, in accordance with one or more aspects of the present disclosure;

FIG. 2N schematically illustrates an embodiment of an air/water cylinder;

FIG. 2O illustrates a sectional view of another embodiment of the channel separator pin of the suction and air/water port connector shown in FIG. 2A with another embodiment of an annular seal, in accordance with one or more aspects of the present disclosure;

FIG. 3A illustrates a side perspective view of another embodiment of a suction air/water port connector, in accordance with one or more aspects of the present disclosure;

FIG. 3B illustrates the embodiment of the suction air/water port connector of FIG. 3A with the knob removed, in accordance with one or more aspects of the present disclosure;

FIG. 3C illustrates a side perspective view of an embodiment of a body assembly of the suction air/water port connector of FIGS. 3A and 3B, in accordance with one or more aspects of the present disclosure;

FIG. 3D illustrates a sectional view of an embodiment of the suction air/water port connector, in accordance with one or more aspects of the present disclosure;

FIG. 3E illustrates a sectional view of an embodiment of the suction air/water port connector in interaction with the air/water cylinder of an endoscope, in accordance with one or more aspects of the present disclosure;

FIG. 3F illustrates another sectional view of an embodiment of the suction air/water port connector in interaction with the air/water cylinder and the suction cylinder of an endoscope, in accordance with one or more aspects of the present disclosure;

FIG. 3G schematically illustrates a sectional view of a portion of the embodiment of the suction air/water port connector of FIG. 3A to 3F interacting with an embodiment of an air/water cylinder of an endoscope, in accordance with one or more aspects of the present disclosure;

FIG. 4A depicts one embodiment of a biopsy connector, in accordance with one or more aspects of the present disclosure;

FIG. 4B illustrates a user positioning the biopsy connector of FIG. 3A on an endoscope, in accordance with one or more aspects of the present disclosure;

FIG. 4C illustrates an exploded view of the biopsy connector of FIG. 3A, in accordance with one or more aspects of the present disclosure;

FIG. 4D illustrates the body of the biopsy connector of FIG. 3A, in accordance with one or more aspects of the present disclosure;

FIG. 4E illustrates the bio receptacle of the biopsy connector of FIG. 3A, in accordance with one or more aspects of the present disclosure;

FIG. 4F illustrates the shuttle plate of the biopsy connector of FIG. 3A, in accordance with one or more aspects of the present disclosure;

FIG. 4G illustrates the shuttle plate retainer of the biopsy connector of FIG. 3A, in accordance with one or more aspects of the present disclosure;

FIG. 5A illustrates one embodiment of an auxiliary port connector, in accordance with one or more aspects of the present disclosure;

FIG. 5B shows an exploded view of the auxiliary port connector of FIG. 4A, in accordance with one or more aspects of the present disclosure;

FIG. 5C shows the cap of the auxiliary port connector of FIG. 4A, in accordance with one or more aspects of the present disclosure;

FIG. 5D shows a cross-section of the body of the auxiliary port connector of FIG. 4A, in accordance with one or more aspects of the present disclosure;

FIG. 5E shows a cross section of the auxiliary port connector of FIG. 4A, in accordance with one or more aspects of the present disclosure;

FIG. 5F shows the lip seal of the auxiliary port connector of FIG. 4A, in accordance with one or more aspects of the present disclosure;

FIG. 5G shows the face seal of the auxiliary port connector of FIG. 4A, in accordance with one or more aspects of the present disclosure; and

FIG. 6 schematically depicts an embodiment of a cleaning system to be used with the disclosed port connectors.

The drawings are meant to depict salient features of the medical device port connectors and are not specifically provided to scale. Corresponding reference characters indicate corresponding parts throughout several views. The examples set out herein illustrate several embodiments, but should not be construed as limiting in scope in any manner.

### DETAILED DESCRIPTION

The following discussion relates to various embodiments of medical device port connectors. It will be understood that the herein described versions are examples that embody certain inventive concepts as detailed herein. To that end, other variations and modifications will be readily apparent to those of sufficient skill. In addition, certain terms may be used throughout this discussion in order to provide a suitable frame of reference with regard to the accompanying drawings. These terms such as “upper”, “lower”, “forward”, “rearward”, “interior”, “exterior”, “front”, “back”, “top”, “bottom”, “inner”, “outer”, “first”, “second”, and the like are not intended to limit these concepts, except where so specifically indicated. The terms “about” or “approximately” as used herein may refer to a range of 80%-125% of the claimed or disclosed value.

Reusable medical devices need to be cleaned and/or disinfected and/or sterilized between uses to prevent cross-contamination and the resulting iatrogenic diseases and nosocomial infections. However, there are many challenges to achieving adequate cleaning and disinfection of reusable medical devices, such as endoscopes that may have narrow channels (lumens) and be made of heat sensitive materials. One challenge in the art has been the formation of seals between cleaning devices and exterior ports of reusable medical devices. Although internal seals have been employed, such systems and methods may obstruct the internal surface area of a reusable medical device during cleaning cycles. Advantageously, one or more of the connectors disclosed herein make seals on the external surface of reusable medical devices, e.g., endoscopes, such that there is less obstruction, and in some embodiments, no obstruction of the internal surface areas of reusable medical devices during cleaning cycles. The following description and corresponding figures describe embodiments of the inventive connectors as used with a reusable endoscope, however it should be noted that one or more of said connectors may be used with other medical devices while providing some or all of the benefits described with regard to the reusable endoscope.

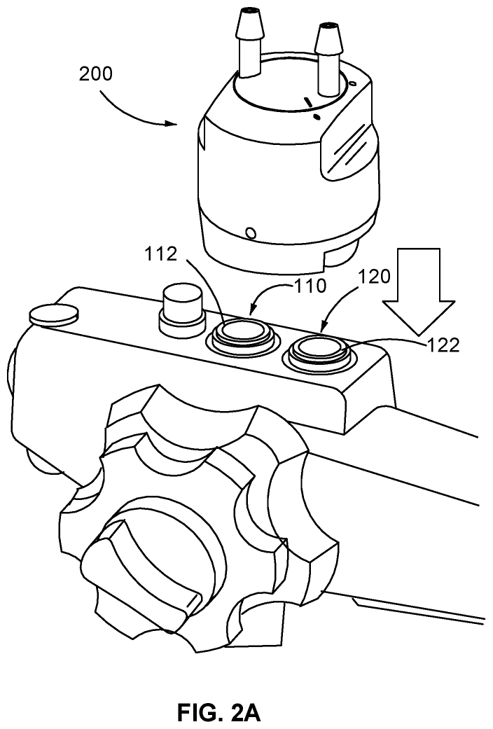

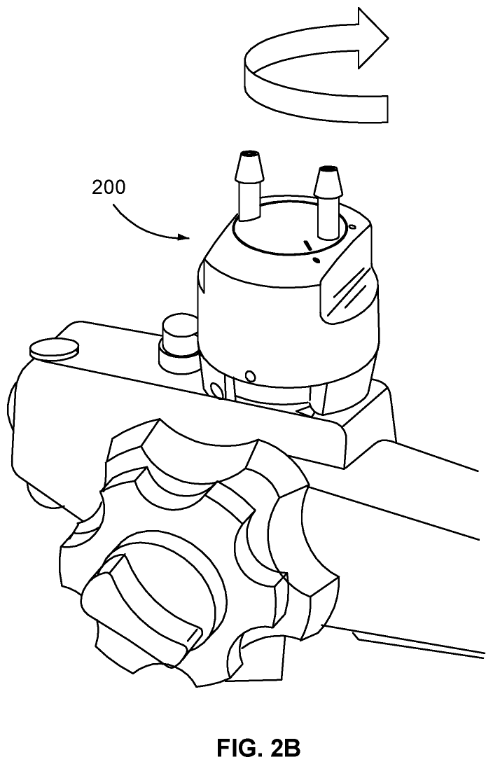

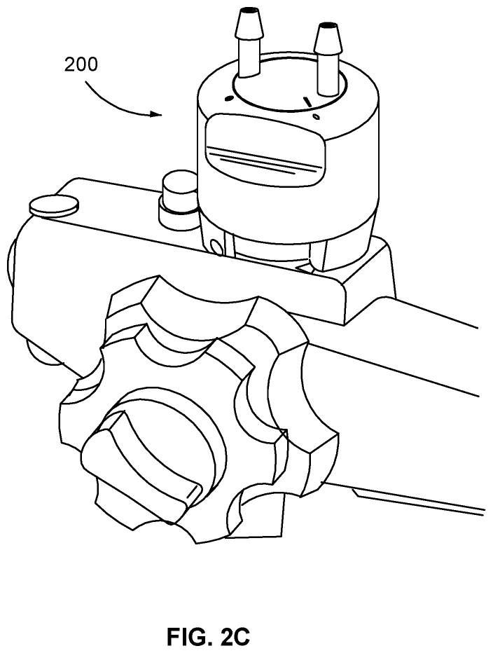

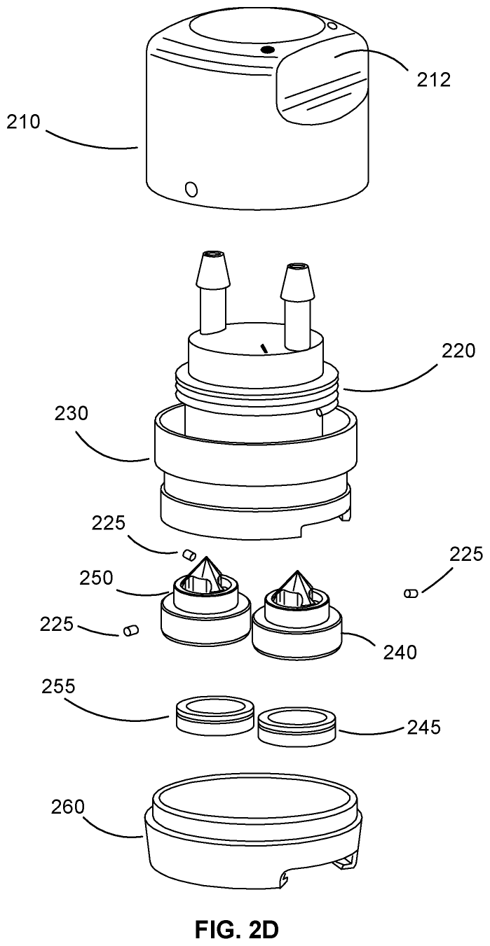

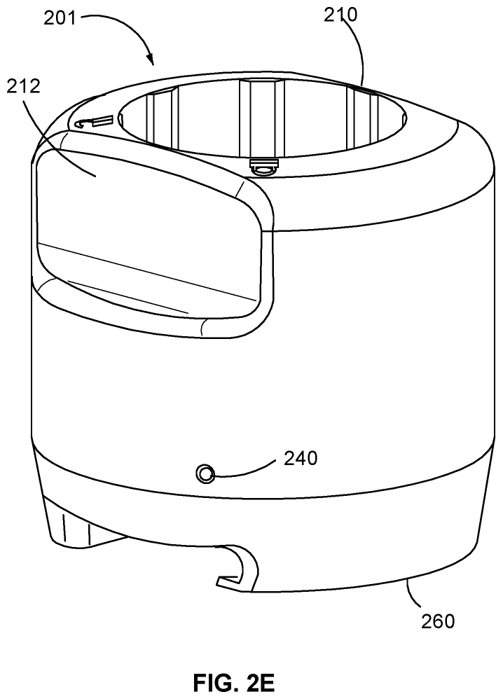

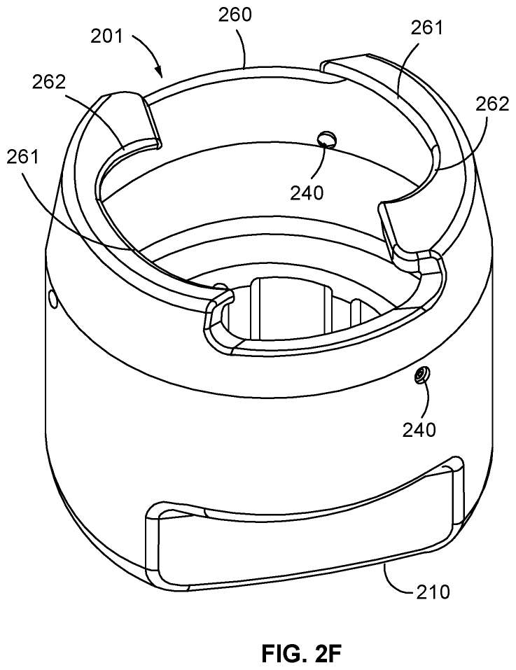

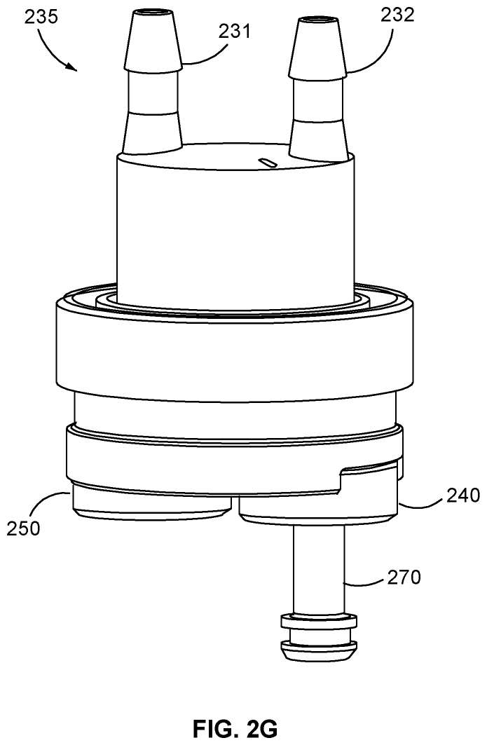

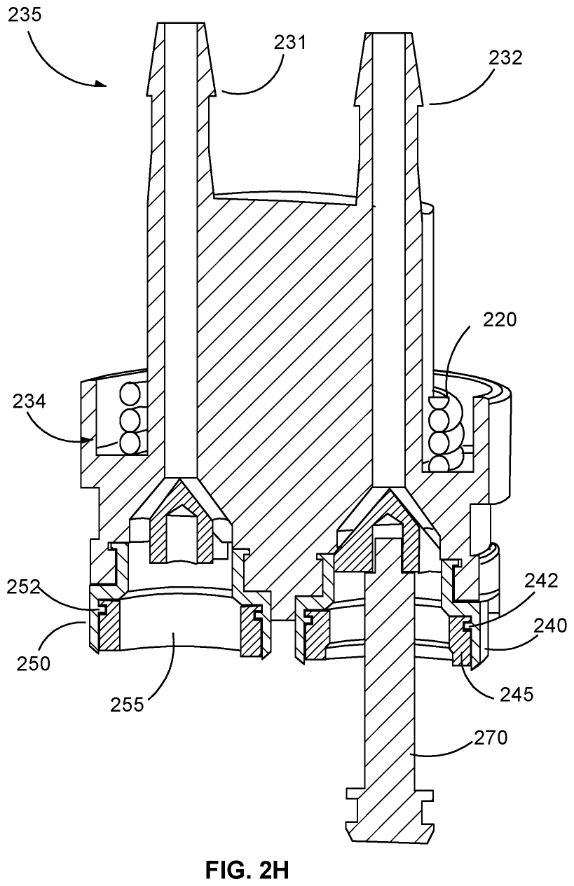

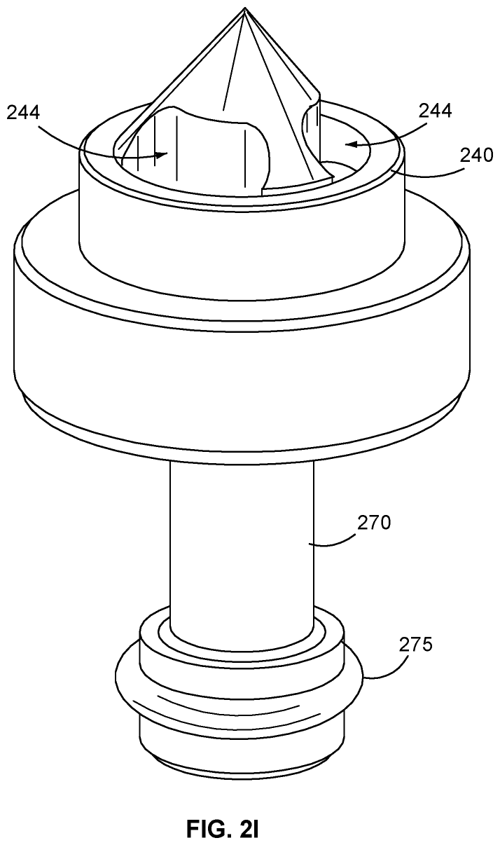

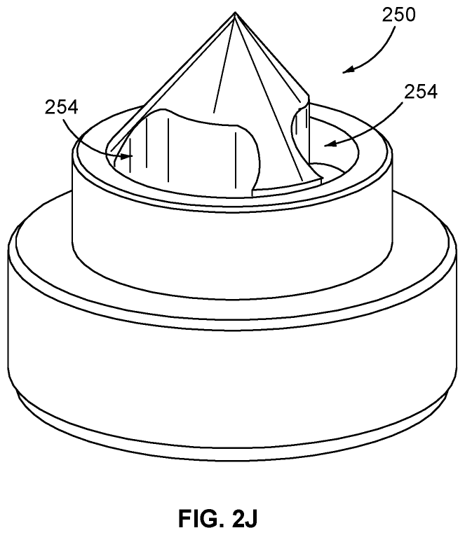

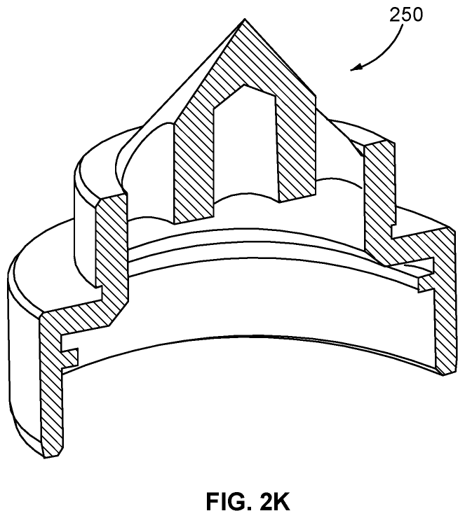

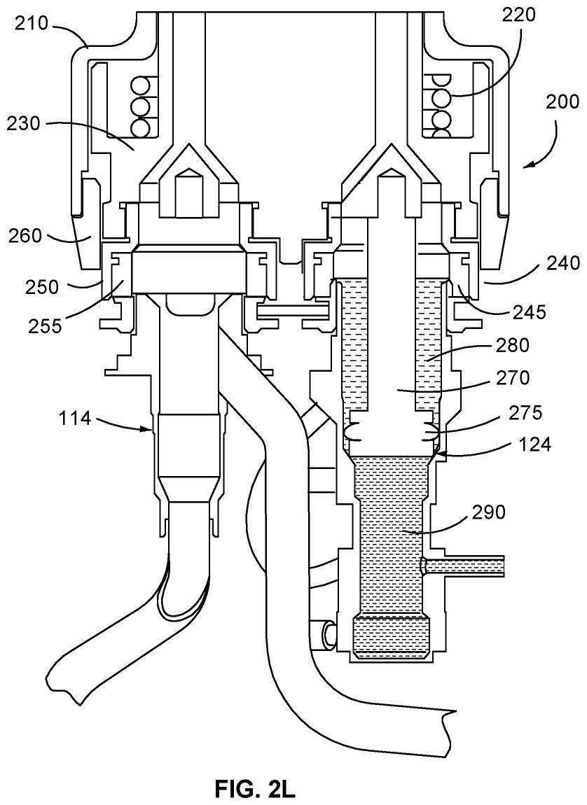

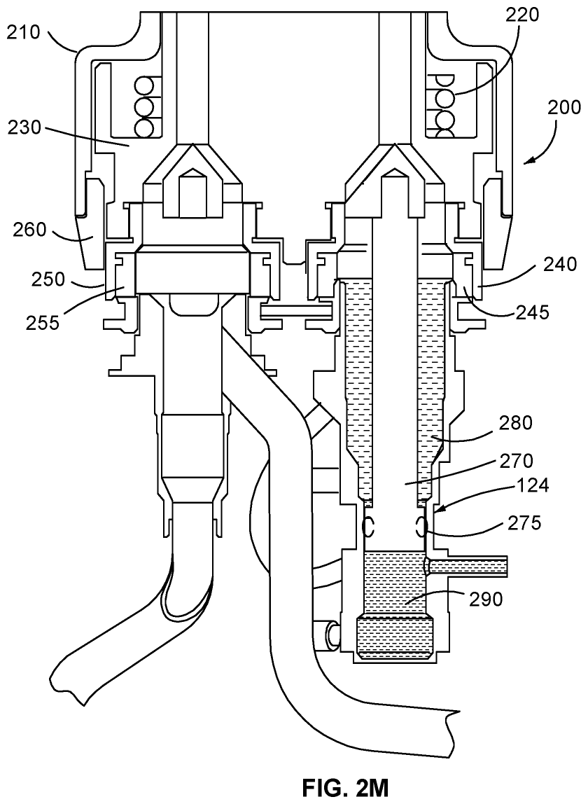

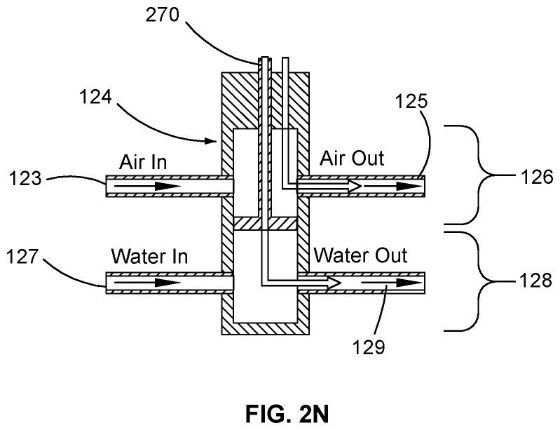

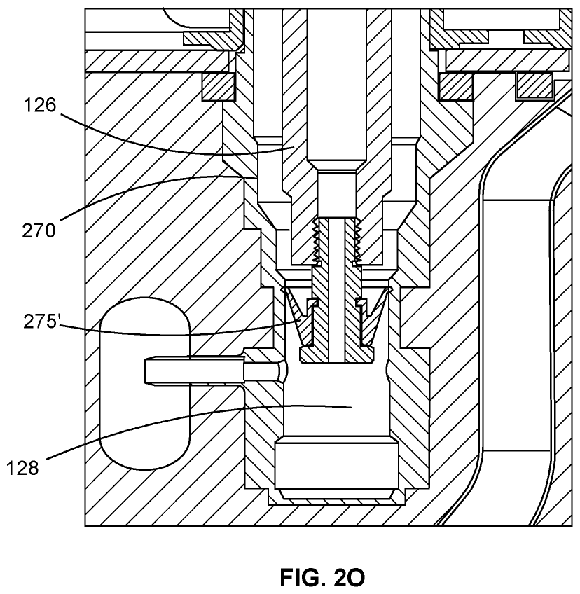

For example, it can be difficult to obtain good seals between the suction cylinder 114 (see FIG. 2L) and air/water cylinder 124 (see FIG. 2L) on the outside surface of an endoscope 100 to pass cleaning fluid through the suction port 110 and the air/water port 120 to the corresponding endoscope channels. Such difficulty arises in part because the suction cylinder 114 (see FIG. 2L) and air/water cylinder 124 (see FIG. 2L) are positioned in close proximity to each other as well as to the control knob and the control button. Additionally, both of these endoscope ports have quite small lips/undercuts to seal and fluidly couple with external connectors to cleaning devices.

It has also been challenging to connect and seal on to endoscope biopsy ports via contacts with the exterior of the endoscope, so that cleaning fluid can be passed through the corresponding endoscope channels during cleaning procedures. Biopsy ports may have a relatively simple geometry with only a circular lip to connect with cleaning devices. Because the internal chamfer on the top lip of the biopsy port makes a small area to form an axial seal, operators have difficulty forming secure fluid connections between biopsy ports and cleaning devices.

Another challenge has been to connect and seal cleaning devices to external endoscope auxiliary ports, so that cleaning fluid can be passed through the corresponding channels to clean the endoscope. The auxiliary port has a relatively simple geometry and may comprise a threaded interface for attachment. A button may be located in close proximity to the auxiliary port. A chamfer feature below the threaded part provides a limited available contact area for axial sealing.

Therefore, there remains a need in the art for endoscope connectors that can seal the suction, air/water, biopsy, and auxiliary ports to cleaning devices, so that adequate cleaning and/or disinfection and/or sterilization can be achieved. The present disclosure relates to connectors to the various ports of reusable medical devices, such as endoscopes.

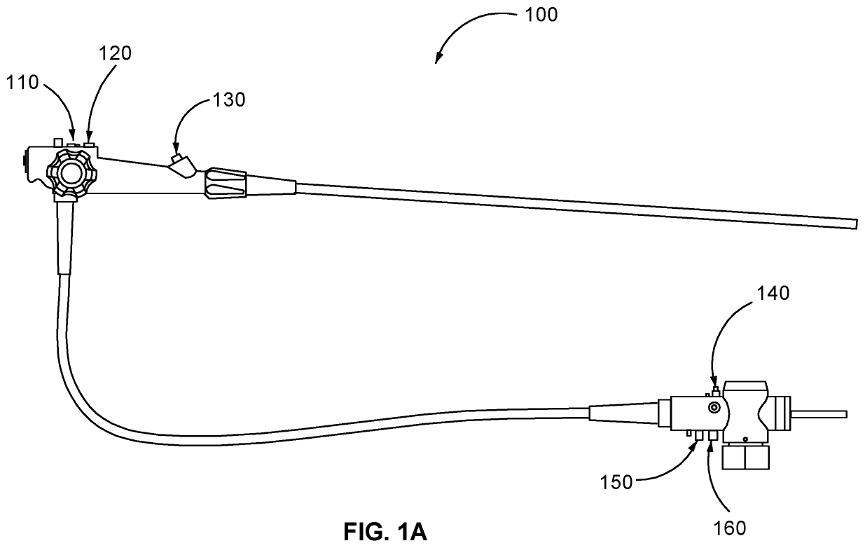

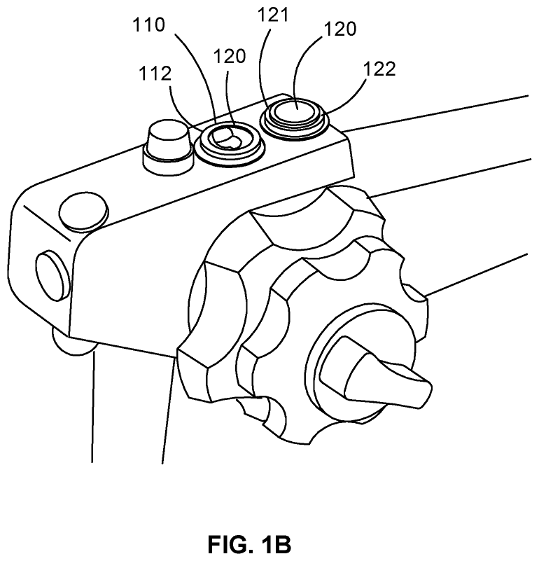

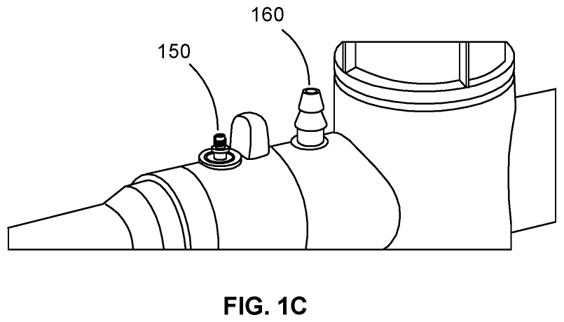

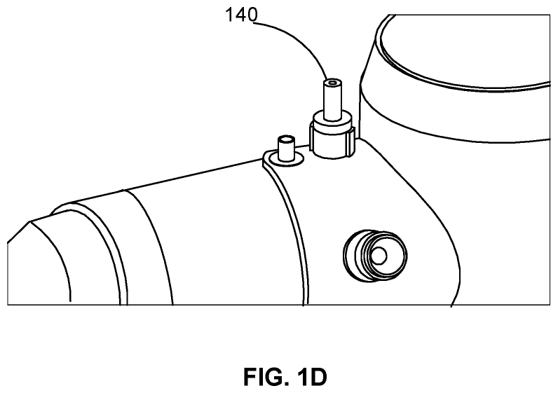

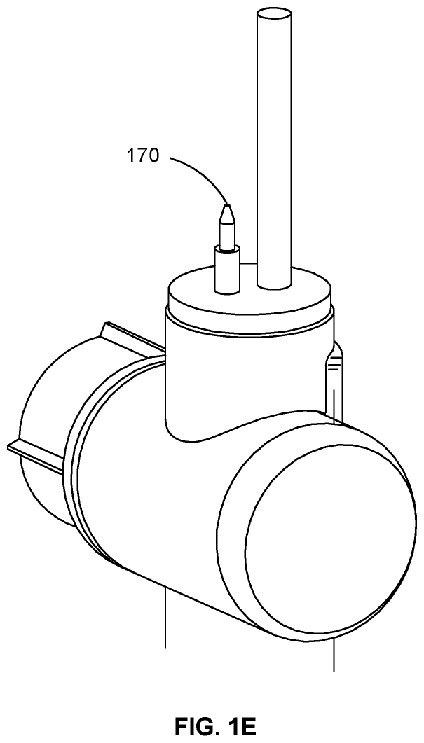

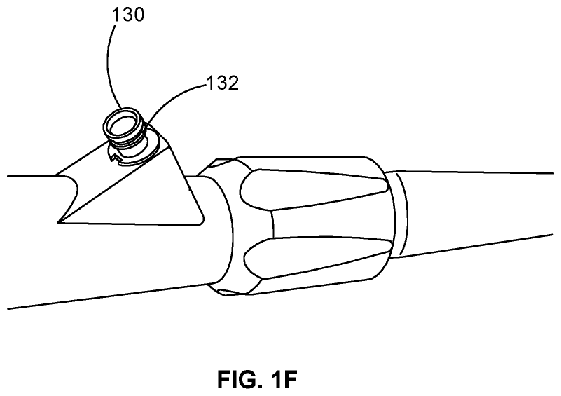

Advantageously connectors designed in accordance with this disclosure implement user-friendly mechanisms that engage with the various endoscope ports. For example, FIG. 1A depicts one embodiment of endoscope 100 comprising suction port 110, suction air/water port or air/water port 120, biopsy port 130, air/water bottle port 140, auxiliary port 150, and suction port 160. A close up view of suction port 110 and air/water port 120 is shown in FIG. 1B. As shown, the suction port 110 and the air/water port 120 define an outer circumferential lip 112, 122. FIG. 1C shows a close up view of auxiliary port 150 and suction port 160. FIG. 1D shows a close up view of air/water bottle port 140. FIG. 1E shows the air-pipe port of endoscope 100. FIG. 1E shows a close up view of the biopsy port 130.

One embodiment of a user-friendly suction and air/water port connector 200 employs a push and twist mechanism where the suction and air water connector is pushed down onto the suction 110 and the air/water port 120 to engage the suction port 110 and the air/water port 120. The suction and air/water port connector 200 is then rotated relative to the medical device (in this case an endoscope) to secure the suction and air/water port connector 200 to the suction port 110 and the air/water port 120. The amount of rotation needed to secure the suction and air/water port connector 200 may vary depending on the medical device, however in some embodiments only a quarter turn is required. Of course, it should be appreciated that connectors can be configured to require any suitable turning extent to secure attachment in accordance with embodiments of the invention—e.g. 60°, 70°, 80°, 100°, 110°, or 120°. In FIG. 2A, the down arrow indicates lowering of one embodiment of a suction and air/water port connector 200 onto suction port 110 and air/water port 120. FIG. 2B illustrates rotation of knob relative to the body of suction and air/water port connector 200 to engage the suction and air/water ports and lock the connector in place. FIG. 2C shows suction and air/water port connector 200 in the locked configuration on an endoscope.

In the embodiment shown in FIG. 2D, the suction and air/water connector 200 comprises a gripping portion 210 such as a knob, locking spring or force generator 220, body 230, fasteners 225, such as screws, air/water seal 245, sealing cups 240 and 250, suction seal 255, and locking ring 260 or locking member (FIG. 2D). Other embodiments of the suction and air/water port connector 200 may include fewer or more components while keeping with the spirit of the invention. It should be appreciated that each part may be made of any suitable material by any suitable manufacturing method. In one embodiment, gripping portion 210 and body 230 are made of plastic, e.g., Acetal/polyoxymethylene (POM), and may be manufactured by any suitable method, e.g., molding and/or machining. In one embodiment, force generator 220 is a locking spring made of SST-302. In one embodiment, screws 240 may be made of off the shelf SST-316. In one embodiment, air/water seal 245 and suction seal 255 comprise silicone, e.g., compression molded silicone rubber (CMSR) and Liquid Silicon Molding. In one embodiment, sealing cups 240 and 250 and locking ring 260 comprise SST-316 and may be machined.

FIGS. 2E and 2F depict knob assembly 201, which comprises a gripping portion 210 and locking ring 260. One embodiment of the gripping portion 210 may comprise one or more grip elements 212 to facilitate gripping and turning during installation and removal of the suction and air/water port connector 200 from an endoscope 100. As shown in FIGS. 2D and 2E, the grip element 212 comprises at least one flat cut-out, however in other embodiments the grip element may include one or more textured portions, one or more ridges or grooves or any other feature configured to aid in the gripping and manipulation of the suction and air/water port connector 200. In one embodiment, locking ring 260 comprises asymmetric compound arc profile, wherein arc profiles 261 on opposite ends of locking ring 260 slide under the circumferential lip 112 of the suction port and the circumferential lip 122 of the air/water port 120 (see FIGS. 1B and 2A) for preliminary engagement and arc profiles 262 at least partially wrap around the circumferential lips 112, 122 and engage when rotated (FIG. 2F). Arc profiles 262 also have end stop features that indicate to a user that the gripping portion 210 has rotated into the locked position. It should be appreciated that similar locking action may be achieved using different profiles, including those that comprise compound curves. The locking ring 260 is configured to inhibit movement of the suction and air/water connector 200 (such as racking or tilting) relative to the endoscope 100 which could result from unequal pressures of cleaning and/or disinfection fluid being introduced into the suction port 110 and the air/water port 120 (see FIGS. 1A and 1B).

FIGS. 2G and 2H depict one embodiment of a main body 235 or main body assembly or body assembly. One embodiment of main body assembly 235 comprises body 230 with two barb features 231 and 232, which aid to secure separate tubing for each cylinder 114, 124 (see FIG. 2L) to pass cleaning and/or disinfection agent into the endoscope. One or more components of the main body assembly 235 may be formed as a single monolithic unit with the main body assembly 235. Body 230 may also comprise taper threads to secure sealing cups 240 and 250 in place. A circular cut out 234 holds the force generator 220 in place. Main body assembly 235 comprises sealing cups 240 and 250, which may optionally comprise a showerhead profile, along with silicone seals 245 and 255. A sealing force is applied by the force generator 220 mounted between knob assembly 201 and main body assembly 235 to the air/water seal 245 and the suction seal 255.

Sealing cups 240 and 250 hold air/water seal 245 and suction seal 255 in place via an undercut/protruding metal flange 242, 252, which grabs onto each seal. The optional showerhead profile of sealing cups 240 and 250 comprises a conic profile on the top and three lozenge features 244, 254 positioned at an angle relative to each other (FIGS. 2I-2K). In the embodiment shown, the three lozenge features are positioned at a 120 degree angle relative to each other. The showerhead profile advantageously allows fluidic flow into the endoscope without obstructions. Specifically, the showerhead profile is structured to minimize fluid impedance through the suction and air/water connector 200 particularly in instances where the cleaning and/or disinfection agent comprises a slurry. For example, some cleaning systems may rely on the kinetic energy of a slurry to effectuate cleaning of a lumen; the disclosed showerhead profiles may act beneficially so as not to impede a flow of this slurry and reduce its kinetic energy. Channel separator pin 270 may optionally be included in main body assembly 235.

Channel separator pin 270 may connect with the air/water sealing cup 240 via a thread engagement or by any other suitable means, such as a press-fit engagement. Channel separator pin 270 separates the air and water channels of an endoscope during a cleaning cycle. Channel separator pin 270 may further comprise annular seal 275, such as an O-ring, a wiper seal and/or a pressure actuated seal, however any suitable sealing element may be used. The annular seal 275 rests inside the surface of the air/water cylinder 124 such that it separates air and water channels, respectively. Two embodiments of channel separations are depicted in FIGS. 2L and 2M, where the location of the annular seal separates air flow 280 and water flow 290. FIG. 2N schematically shows the air/water cylinder 124 with the channel separator 270 in place. As can be seen, the air/water cylinder 124 essentially comprises a first portion 126 and a second portion 128. The first portion includes an air inlet 123 and air outlet 125 and the second portion 128 includes a water inlet 127 and a water outlet 129. Separation of the first portion 126 from the second portion 128 by the channel separator 270 is required to ensure a flow of cleaning fluid through each portion 126, 128 simultaneously. Referring to FIG. 2O, a different type of annular seal 275′ is depicted that is configured to seal against an inside of the air/water cylinder 124 as previously described, however, in the case where the pressure in the first portion 126 is less than the pressure in the second portion 128, the annular seal 275′ is configured to flex. The flexing enables the portion of the air/water cylinder 124 originally covered by the annular seal 275′ to be cleaned. While specific configurations have been described and illustrated, it should be appreciated that suitable variations can be implemented depending on the configuration of the air/water cylinder 124.

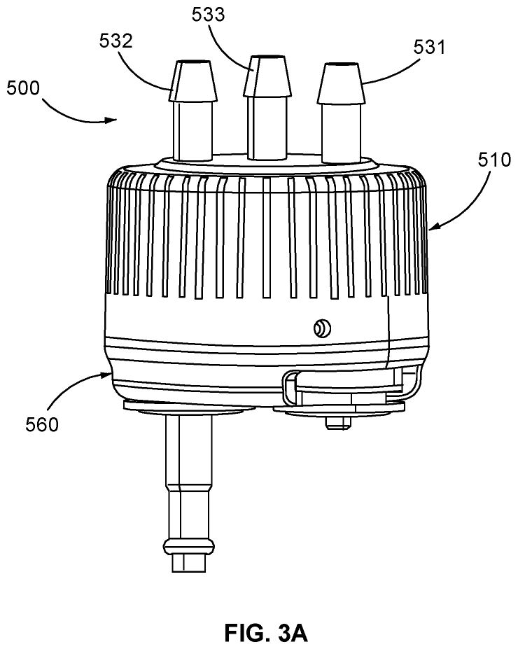

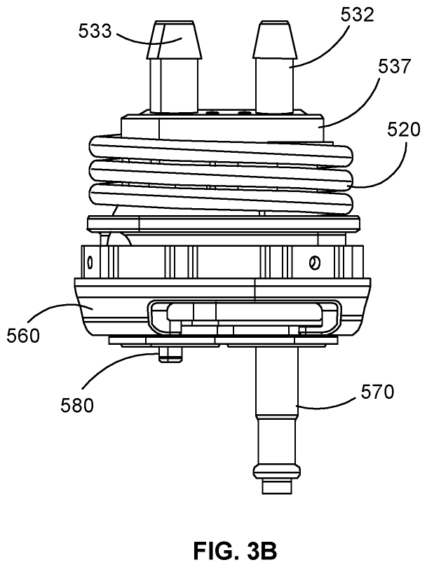

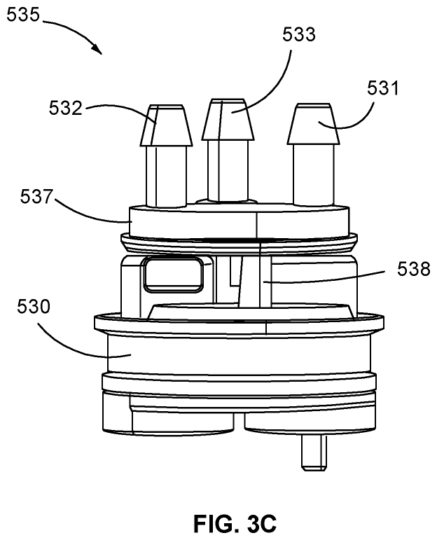

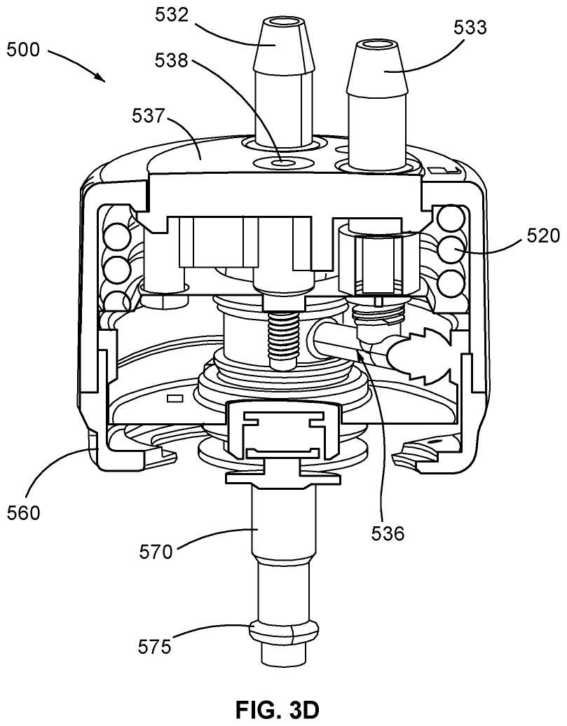

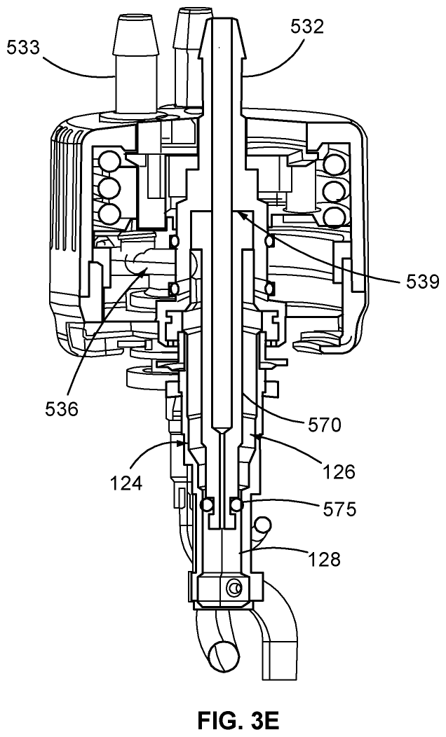

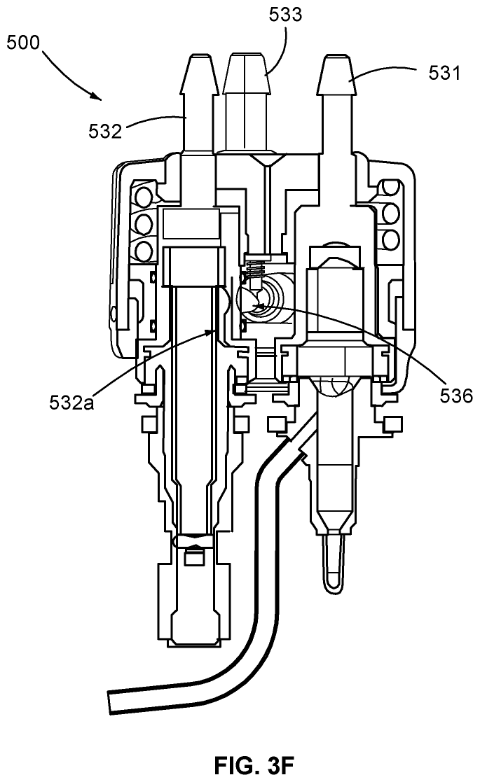

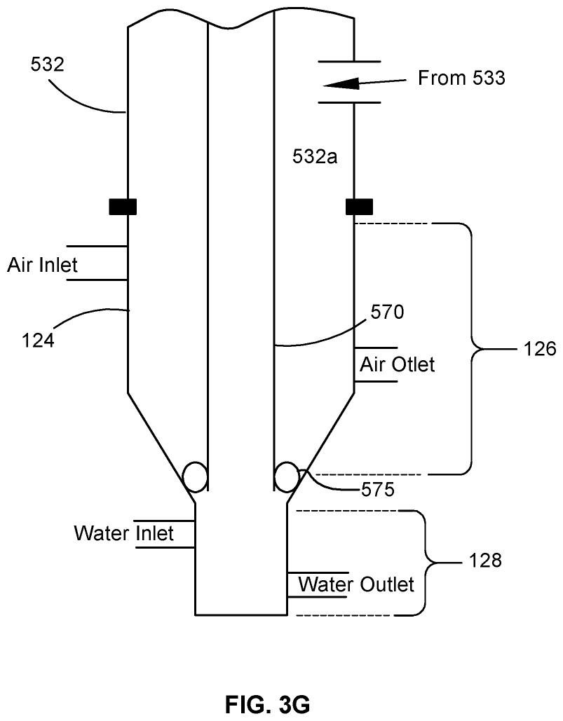

Another embodiment of the suction air/water port connector 500 will be discussed with reference to FIGS. 3A to 3G. Similar to the other embodiments discussed, the air/water port connector 500 employs a similar push and twist mechanism as previously described to engage the suction port 110 and the air/water port 120. The suction and air/water port connector 500 generally comprises knob 510, force generator 520 such as a locking spring to apply a sealing force, a body assembly 535 and a locking ring 560 coupled to the body assembly 535. The force generator 520, knob 510 and the locking ring 560 are configured similar to previously discussed embodiments and perform in a similar manner. As in the previously described embodiments of the suction air/water port connector 200, the main body assembly 535 comprises body 530, however in this embodiment of the port connector 500, the body 530 comprises three barb features 531, 532 and 533 which aid to secure separate tubing for each cylinder 114, 124 (see FIG. 2L) to pass cleaning and/or disinfection agent into the endoscope 100. As shown in FIG. 3C, a body cap 537 surrounds a portion of each of the barbs 531, 532, 533 and is coupled to the body 530 with a coupling member 538, such as a screw. The aspects of the suction air/water port connector 500 are the same as previously described embodiments as they relate to the cleaning of the suction cylinder through barb 531 and will not be described. However, barb 531 now includes a pin 580. The pin 580 helps ensure that the rotational motion of the suction air/water port connector 500 is maintained along a central axis of rotation.

Turning to FIG. 3D it can be seen that barb 533 is fluidly connected with barb 532 via a conduit 536 defined in the body 530. In this manner, barbs 532 and 533 are configured to fluidly couple to a cleaning system to receive cleaning fluid to be dispensed in to the air/water cylinder 124. Similar to other embodiments of the suction air/water connector 200 previously described, this embodiment also includes a channel separating pin 570 with an annular seal 575. However, referring to FIGS. 3E and 3F, the channel separating pin 570 is not connected to a sealing cup and is instead coupled to an end 539 of the barb 532 opposing the fluid connection to the cleaning system. The channel separating pin 570 is sealed against an inner surface of the air/water cylinder 124 to separate the first portion 126 and the second portion 128 of the air/water cylinder 124. The channel separating pin 570 delivers cleaning fluid directly to the second portion 128 of the air/water cylinder. Cleaning fluid is additionally introduced through the third barb 533, through the conduit 536 and into an area 532a between the second barb 532 and the channel separating pin 570 to deliver cleaning fluid to the first portion 126 of the air/water cylinder 124. This is also shown schematically in FIG. 3G. In this embodiment of the suction air/water port connector 500, the pressure in the first and second portions 126, 128 of the air/water cylinder 124 can be adjusted separately to prevent pressure build-ups in the peripheral endoscopy tubing, which could decrease flow velocity of the cleaning fluid and/or damage the equipment. It should be appreciated that each part may be made of any suitable material by any suitable manufacturing method similar to the embodiments already discussed. While specific configurations have been described and illustrated, it should be appreciated that suitable variations can be implemented.

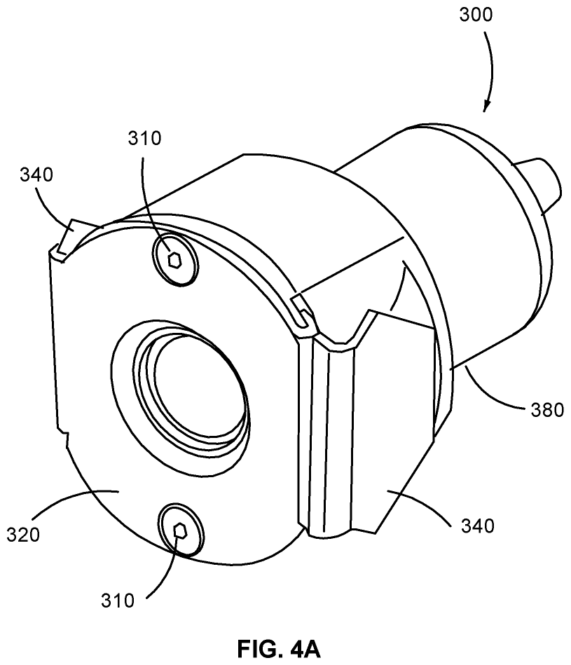

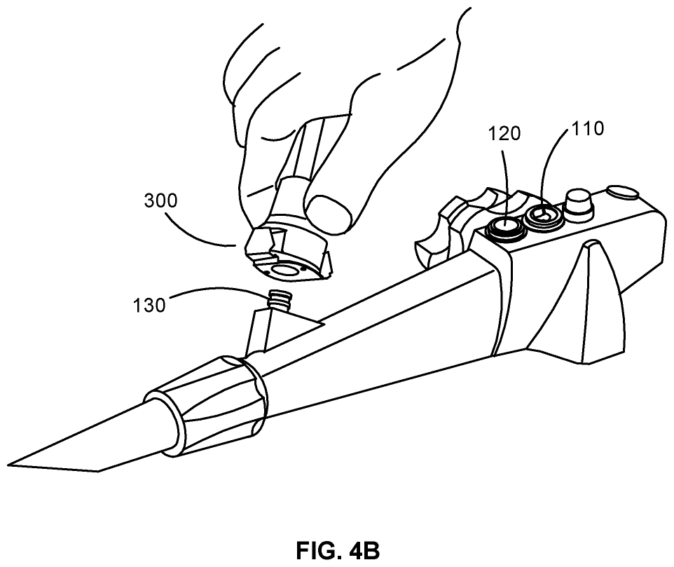

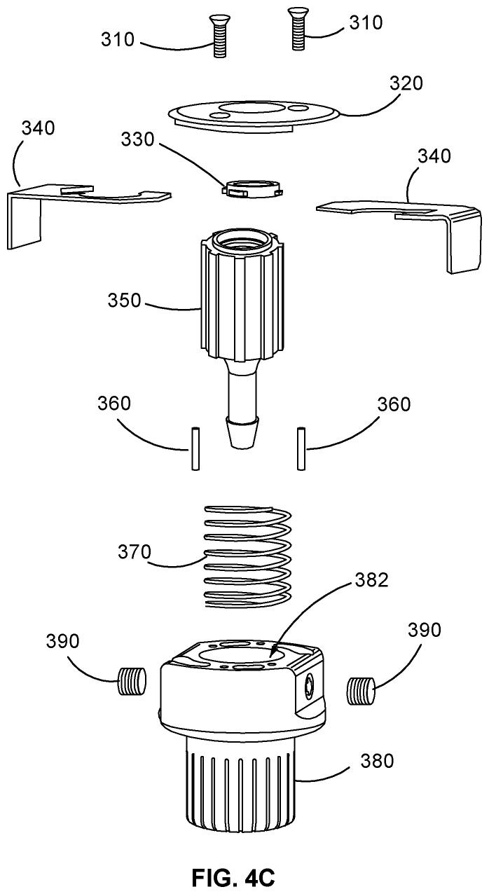

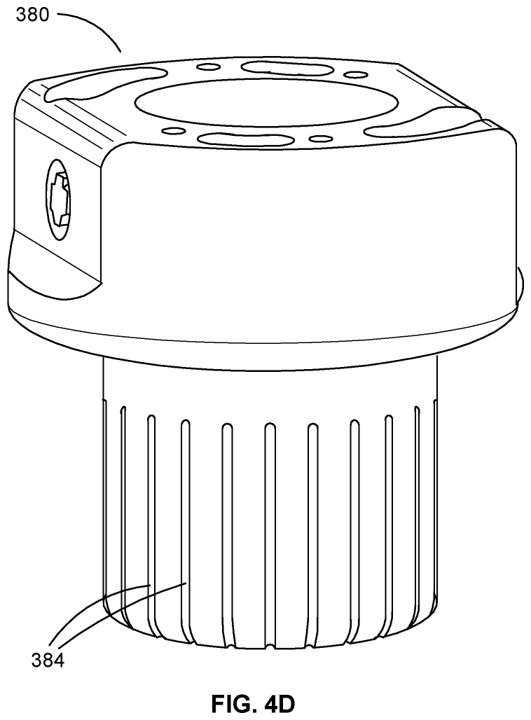

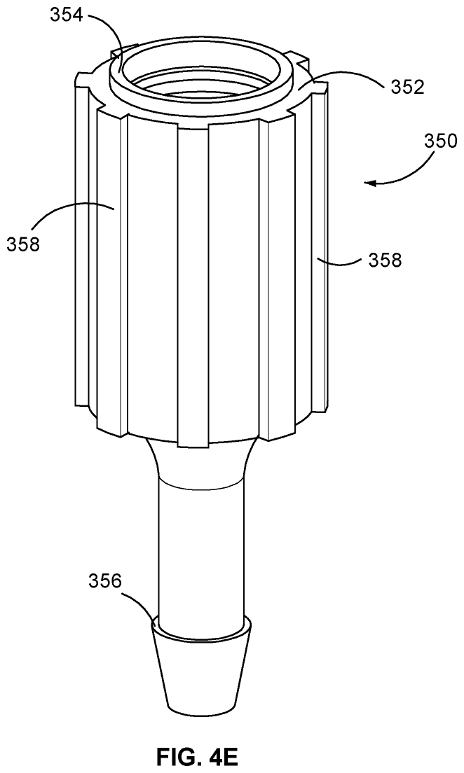

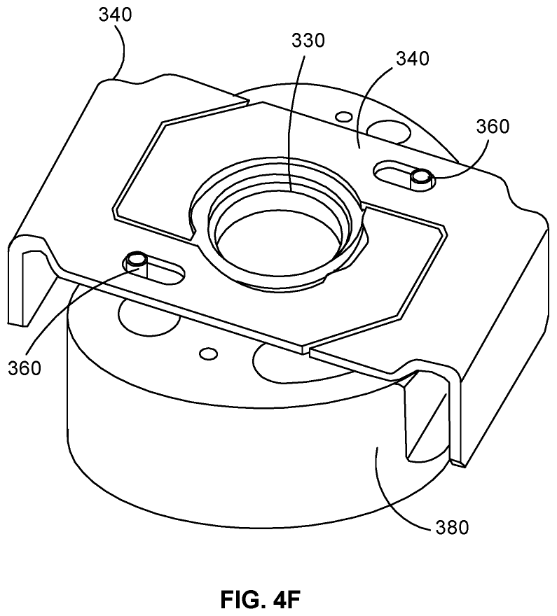

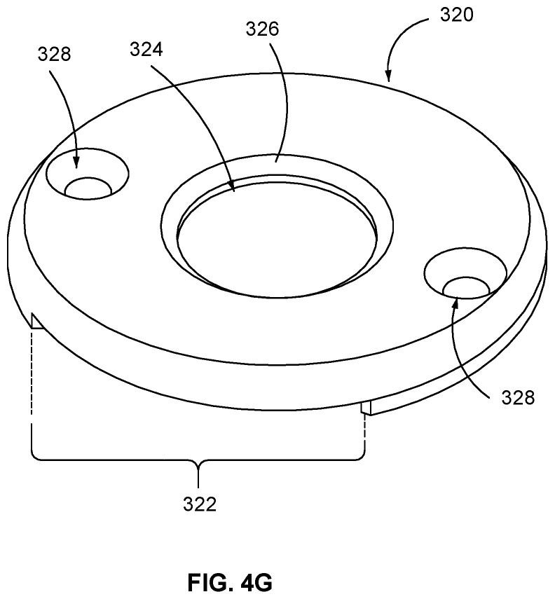

The disclosure further relates to connectors that provide a user-friendly way to couple a cleaning device to the biopsy port, e.g., a push and latch mechanism. FIGS. 4A to 4G illustrate various aspects of one embodiment of biopsy port connector 300. In one embodiment biopsy connector 300 comprises a dual shuttle design. In one embodiment, biopsy connector 300 comprises housing/body 380 including one or more gripping elements 384 (e.g., a bio slide grip comprising machined/molded acetal), bio receptacle 350 (e.g., machined/molded acetal), which holds seal 330 (e.g., molded silicone) in position and backed up by a force generator 370 (e.g., SS302 compression spring) to provide sealing force, two shuttle plates 340 (e.g., laser cut and folded SS316) retained via shuttle plate retainer 320 (e.g., machined SS316) for engagement and disengagement of biopsy connector 300 (FIG. 4C). Biopsy connector 300 may further comprise on or more fasteners 310 (e.g., SS316 M2×8 Csk screws), dowel pins 360 (e.g., SS316), and biasing members 390 such as shuttle springs (e.g., SS302). Although exemplary materials and methods of manufacture are disclosed, it should be appreciated that any suitable materials and manufacturing processes may be employed.

Barb geometry on bio receptacle 350 allows separate tubing to connect and pass cleaning agent into the endoscope. Biopsy connector 300 may employ a spring and shuttle mechanism where, during biopsy connector engagement, biopsy port connection on the endoscope engages with bio receptacle 350, which accommodates the seal. As the user pushes the connector on the endoscope, bio receptacle 350 overcomes the spring compression force and moves back inside housing/body 380, and shuttle plates 340 to the locking position via shuttle plate force generator 370 (FIG. 4B). To disengage biopsy connector 300 from the endoscope biopsy port, a user depresses both shuttle plates 340 at once.

In one embodiment, housing/body 380 is made of a suitable material, e.g., plastic, and comprises a blind hole 382 centrally positioned or positioned in the middle to accommodate bio receptacle body 350 which is backed up by compression spring (FIG. 4D). Two dowel holes placed diagonally control shuttle plate 340 movement, during engagement and disengagement. Two threaded holes may be provided for fasteners 310 to secure shuttle plate retainer 320 or retainer plate in place. Holes in housing/body 380 accommodate shuttle plate biasing members 390. Biasing members 390 enable shuttle plates 340 to move back and forth during engagement and disengagement. Optionally, housing/body 380 may comprise dummy core outs to reduce the weight of the biopsy connector 300 and are manufacturing process friendly.

As shown in FIG. 4E, bio receptacle 350 may comprise a plastic body which has an undercut feature 352 to hold the seal 330 in place. In another embodiment, the seal 330 may be overmoulded silicone onto the bio receptacle 350 using any known overmoulding process such as CMSR and LSR) A protruding rim 354 in the front face and proximate the undercut feature 352 acts as stopper for shuttle plates 340 during engagement and disengagement. A barb feature 356 may be provided to secure and fluidly couple to tubing of a cleaning device in order to pass cleaning and/or disinfection fluids. In one embodiment, a plurality of ribs 358 or guides are defined on the external surface of bio receptacle 350 and act to guide bio receptacle 350 to slide and be properly positioned inside housing/body 380. The plurality of guides 358 decrease the surface contact between the bio receptacle 350 and the housing/body 380 to provide a low co-efficient of friction between the two components as well as to reduce the weight of the bio receptacle 350. In another embodiment, the bio receptacle does not include a plurality of guides 358.

Shuttle plates 240 may comprise stainless steel formed sheet metal (FIG. 4F). In one embodiment an internal profile drives the engagement and disengagement of the biopsy connector and a slot controls the travel of shuttle plates 340. Advantageously, shuttle plate 340 may be designed for symmetric assembly and are mirror images of each other. Advantages of shuttle plates according to the disclosure include ease of manufacturability. Additionally, the use of a common part to achieve locking/unlocking action can be mirrored in assembly. In one aspect, a dual shuttle plate mechanism provides ample latching area under biopsy port lip and exerts a radial compressive force against the biopsy port or port lip 132 (see FIG. 1F).

Referring to FIG. 4G, the shuttle plate retainer 320 may comprise a stainless steel body with cut-outs 322 on the rear face to accommodate sliding of shuttle plates 340 during engagement and disengagement (FIG. 4G). A through hole 324 with an optional lead in chamfer 326 allows the biopsy port 130 (see FIG. 1) of the endoscope to guide/align with biopsy connector 300. In one embodiment, shuttle plate retainer 320 comprises guide holes 328 for CSK screws to latch onto housing/body 380. Contemplated embodiments further comprise alternatives and variations of these configurations that can be implemented in connectors according to the disclosure.

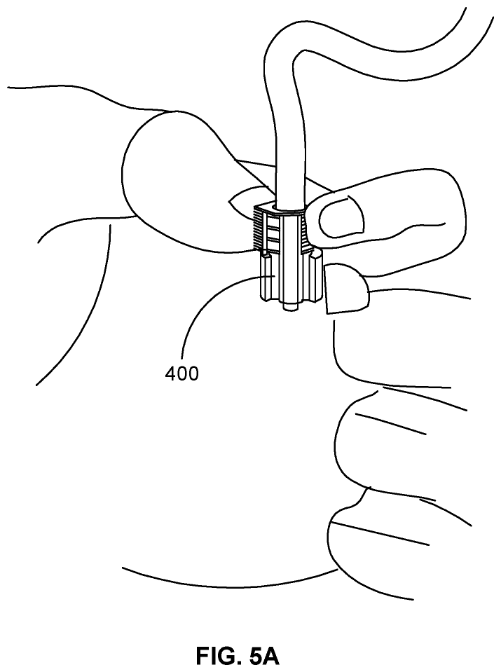

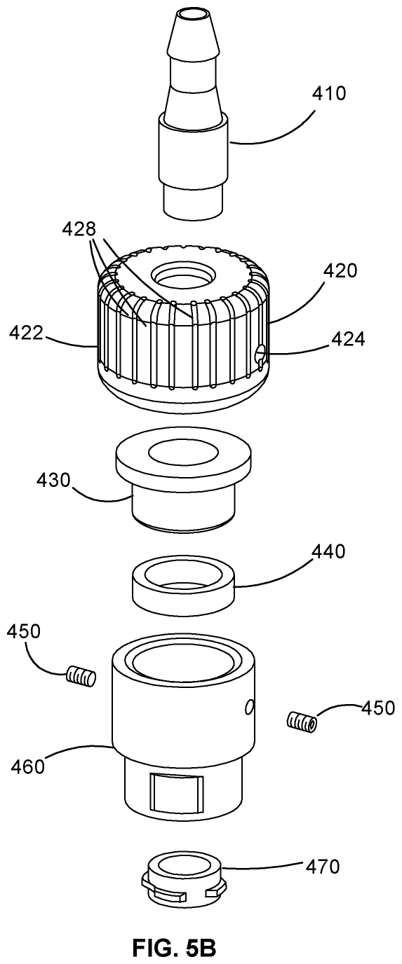

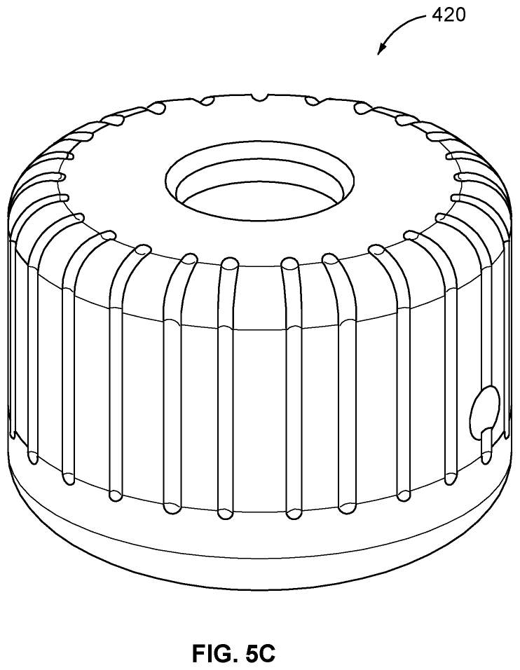

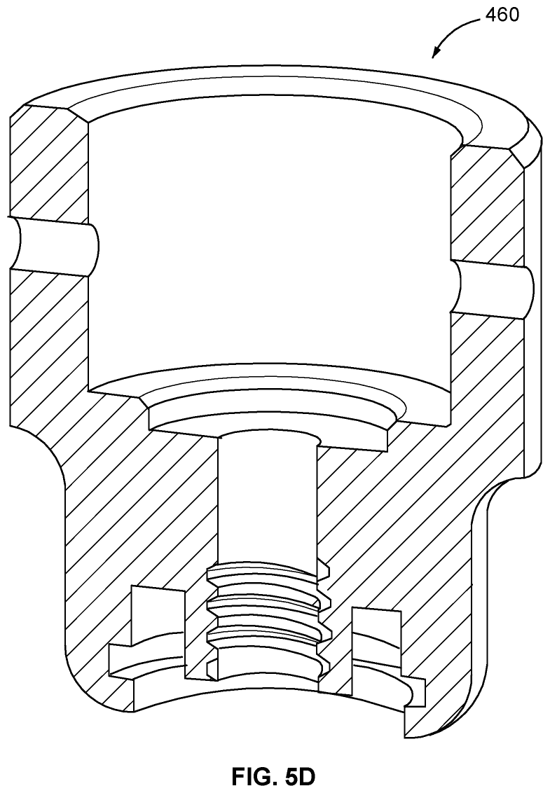

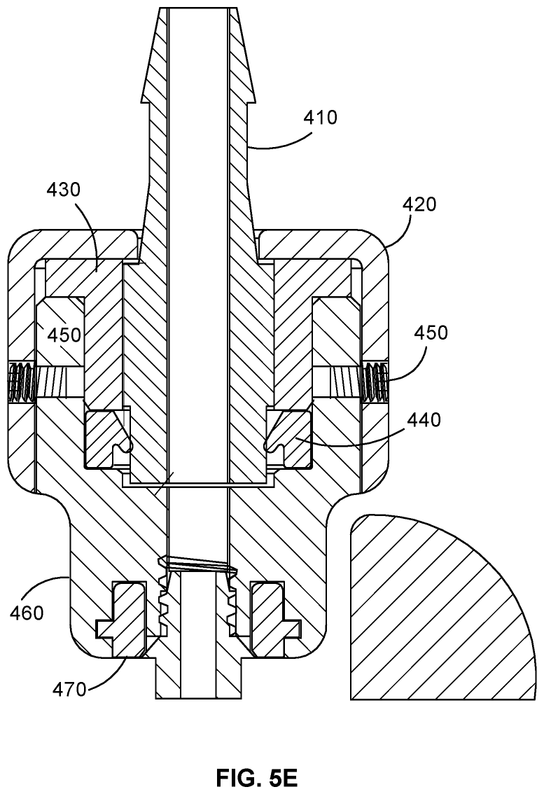

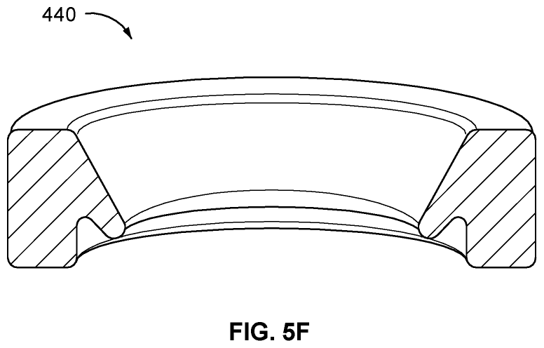

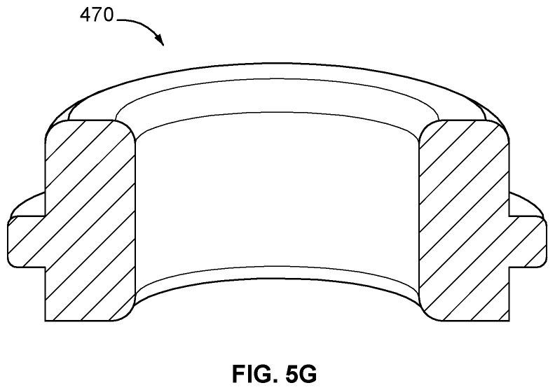

One embodiment of an auxiliary port connector 400 is shown in FIGS. 5A through 5G. In one embodiment, auxiliary port connector 400 comprises a swivel type auxiliary connector configuration that provides a low friction, low dead volume system, which enables swiveling, preventing the coupling tube from twisting and disengagement/leakage. Auxiliary connector 400 comprises a cap 420 (optionally plastic) secured onto a housing 460 (optionally stainless steel) via two grub screws 450 (e.g., off the shelf SS316). The housing 460 engages or otherwise holds a face seal 470 on the bottom of the connector which engages with the auxiliary port 150 (see FIG. 1) during the locking phase of connecting auxiliary port connector 400 to the auxiliary port 150 (see FIG. 1) of the endoscope 100. Radial lip seal 440 is positioned or held at partially within the housing 460, and is pressure activated. The barb 410 is configured to fluidly connect the endoscope channel cleaner device to an endoscope 100 in order to pass cleaning fluid.

The connector is driven via the threaded feature on to the auxiliary port, where during connector engagement phase the auxiliary connector is rotated or screwed onto the auxiliary port to gain solid attachment. During the final engagement phase, the face seal 470 on the bottom half of the auxiliary port connector 400 seals onto the chamfer feature below threaded part of the auxiliary port 150 (see FIG. 1). On the other hand during disengagement phase, user just rotates in an opposite direction or unscrews the connector form the auxiliary port 150 (see FIG. 1).

One embodiment of cap 420 (FIG. 5C) comprises molded plastic and acts as an outer cover for auxiliary connector 400 and barb 410 (e.g., machined SS316) and bushing 430 (e.g., off the shelf Iglide®) in place. Two holes 426 defined on the side 422 enable the cap 420 (e.g., molded acetal/POM) to latch onto housing 460. An optional external grip elements 428, such as a plurality of channels or ridges formed in the cap 420, enables the user to connect and disconnect the auxiliary connector smoothly. In another embodiment, the auxiliary port connector 400 includes housing 460 configured to couple and decouple to the auxiliary port 150 and a floating barb 410 configured to swivel independent of the housing 460. This embodiment of the auxiliary port connector 400 may further include an external casing which acts as a bushing and co-axially aligns housing 460 and the barb 410.

One embodiment of housing 460 comprises a stainless steel body (e.g., machined/molded SS316) with a threaded feature in the middle that allows auxiliary connector 400 to screw onto the endoscope auxiliary port and make a secure attachment (FIG. 5D). A bottom section of housing 460 holds seal 470, which engages onto the chamfer feature below a thread part of the auxiliary port. A section of housing 460 holds lip seal 440 (e.g., molded silicone 60 Shore A), barb 410, and bushing 430 and is configured to allow barb 410 to swivel freely during engagement and disengagement (FIG. 5E).

Seals 440 ad 470 are illustrated in FIGS. 5F and 5G, respectively. Face seal 470 (e.g., molded silicone 60 Shore A) engages onto the chamfer feature below a threaded part of the auxiliary port 150 (see FIG. 1). In one embodiment, lip seal 440 is pressure activated, and is activated during the cleaning cycle to seal onto the barb 410. Advantageously, during engagement and disengagement phases lip seal 440 provides minimal friction with barb 410, allowing it to swivel freely.

Barb 410 is held via bushing 430 (e.g., off-the-shelf IGUS brushing). A low coefficient of friction of bushing material (e.g., Iflide®) allows barb 410 to swivel freely during engagement and disengagement. It should be appreciated that any suitable materials and manufacturing processes may be employed and that the exemplary materials disclosed are non-limiting.

The connectors disclosed herein provide numerous advantages including the ability to form secure connections and seals that allow cleaning fluid to be passed through reusable medical devices, such as endoscopes, and avoid problems with leakage and pressure loss in a user friendly way. The disclosed embodiments of connectors are able to withstand fluid pressures of at least 24 psi.

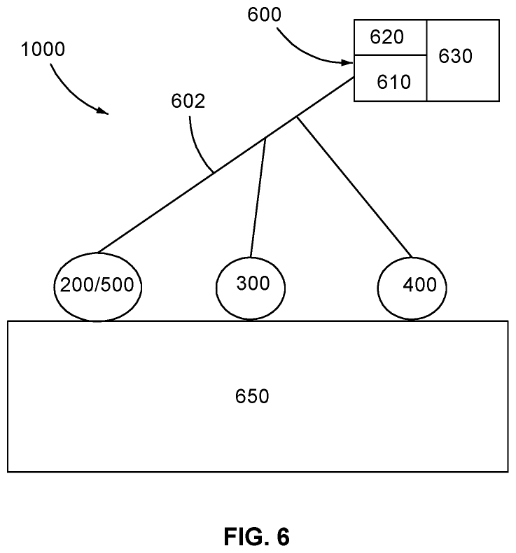

Referring to FIG. 6, the disclosed port connectors 200, 300, 400, 500 may be part of a medical device cleaning system 1000 that comprises a cleaning unit 600 including a cleaning fluid reservoir 610 fluidly coupled to one or more tubes 602 configures to fluidly connect to each of the port connectors 200, 300, 400, 500 to deliver cleaning fluid to said port connectors 200, 300, 400, 500. A pump 620 is configured to control a flow speed of the cleaning fluid and a control unit 630 is configured to enable control of the cleaning unit 600. The cleaning unit 600 may be a self-contained unit or may be comprised of a plurality of individual components. The control unit 630 may be configured to run one or more cleaning cycles to clean the medical device 650 that is connected to the system 1000 via the connectors 200, 300, 400, 500. In an embodiment, a plurality of cleaning programs are stored in the control unit 630 and can be selected by the user. Once the cleaning cycle is completed, the user may be notified by an alarm indicating that it is safe to decouple the medical device 1050 from the system 1000.

While the present invention has been particularly shown and described with reference to certain exemplary embodiments, it will be understood by one skilled in the art that various changes in detail may be effected therein without departing from the spirit and scope of the invention that can be supported by the written description and drawings. Further, where exemplary embodiments are described with reference to a certain number of elements, it will be understood that the exemplary embodiments can be practiced utilizing either less than or more than the certain number of elements.

1. A suction and air/water port connector for a medical device, comprising:
a body assembly;
a gripping portion coupled to the body assembly;
a force generator configured to be at least partially seated within the body assembly;
one or more sealing cups at least partially housed within the body assembly; and
a locking member operatively coupled to the body assembly and configured to removably couple to an exterior surface of a suction port and an air/water port of the medical device to define a locked position and an unlocked position,
wherein in the locked position, movement of the body assembly relative to the suction port and the air/water port is inhibited and a locking force is exerted by the force generator in an axial direction against the suction port and the air/water port to create a liquid-tight seal against the suction port and the air/water port, and
wherein in the unlocked position, the locking member is configured to be removed from the suction port and the air/water port.

2. The suction and air/water port connector of claim 1, further comprising a channel separator pin configured to be at least partially inserted into the air/water port to separate an air channel from a water channel.

3. The suction and air/water port connector of claim 2, wherein the channel separator pin comprises a pressure actuated seal configured to seal the channel separator pin inside the air/water port at a position between the air channel and the water channel.

4. The suction and air/water port connector of claim 1, wherein the locking member comprises an arc profile, and wherein when in the locked position, the arc profile is configured to at least partially surround the suction port and the air/water port.

5. The suction and air/water port connector of claim 1, wherein the one or more sealing cups defines three lozenge features.

6. The suction and air/water port connector of claim 5, wherein the three lozenge features are positioned at an angle relative to each other.

7. The suction and air/water port connector of claim 1, wherein the body assembly comprises at least two barbs.

8. The suction and air/water port connector of claim 1, further comprising at least two seals, and wherein in the locked position, one of the at least two seals is configured to seal against the suction port and another of the at least two seals is configured to seal against the air/water port.

9. A method of manufacturing a suction and air/water port connector for a medical device, comprising:
structuring a body assembly to,
couple to a gripping portion,
at least partially surround a force generator configured to generate a locking force, and
at least partially house one or more sealing cups;
structuring a locking member to operatively couple to the body assembly and to removably couple to an exterior surface of a suction port and an air/water port of the medical device to define a locked position and an unlocked position; and
exerting the locking force in an axial direction against the suction port and the air/water port to create a liquid-tight seal against the suction port and the air/water port when in the locked position.

10. The method of claim 9, further comprising structuring a channel separator pin to be at least partially inserted into the air/water port to separate an air channel from a water channel.

11. The method of claim 10, further comprising structuring the channel separator pin to comprise a pressure actuated seal configured to seal the channel separator pin inside the air/water port at a position between the air channel and the water channel.

12. The method of claim 9, further comprising structuring the locking member to comprise an arc profile to at least partially surround the suction port and the air/water port.

13. The method of claim 9, further comprising structuring the one or more sealing cups to define three lozenge features.

14. The method of claim 9, further comprising structuring the body assembly to comprise at least two barbs to pass fluid through the suction port and the air/water port.

15-27. (canceled)
